(19) United States
(12) Patent Application Publication
(10) Pub. No.: US 2009/0234203 A1
(43) Pub. Date: Sep. 17, 2009

(54) CAPSULE FOR MEDICAL USE

(75) Inventors: Takashi Arita, Shinagawa (JP); Satoshi Sakurai, Shinagawa (JP); Shigemi Kurashima, Shinagawa (JP); Masahiro Yanagi, Shinagawa (JP)

Correspondence Address: IPUSA, P.L.L.C, 1054 31ST STREET, N.W., Suite 400, Washington, DC, 20007, US

(73) Assignee: FUJITSU COMPONENT LIMITED

(21) Appl. No.: 12/400,009

(22) Filed: Mar. 9, 2009

(30) Foreign Application Priority Data
Mar. 14, 2008 (JP) 2008-066549
Aug. 13, 2008 (JP) 2008-208534

(51) Int. Cl. A61B 5/07

(52) U.S. Cl. 600/302

(57) ABSTRACT

A capsule for medical use which is to be passed through a tract of a human or animal body includes a capsule-shape case, a wire antenna placed in a wrapped or folded state on at least a portion of an outer surface of the case, and a fixing member configured to fix the wire antenna in the wrapped or folded state, wherein the fixing member is configured to release the antenna inside the tract.

### BACKGROUND OF THE INVENTION

1. Field of the Invention

The disclosures herein relate to a capsule for use with a medical device for performing medical tests, treatments, and procedures wherein the capsule is designed to pass through a lumen of a human or animal body.

2. Description of the Related Art

A capsule designed to pass through a tract of a human or animal body has been studied for use with a medical device for performing medical tests, treatments, and procedures. Such a capsule has an electronic device (e.g., a camera and a communication unit) embedded therein for use in medical tests. The capsule is swallowed and passed through the gastrointestinal tract by the peristaltic movements of the stomach and intestines for discharge to outside the body.

Pictures of the digestive tract are taken and the image data are transmitted while the capsule is inside the body. In consideration of this, the design and structure of the capsule have been improved over and over again by taking into account hermetic sealing, easiness to swallow, easiness to be propelled, and the like (see Japanese Patent Application Publication No. 2003-135387, for example).

A related-art capsule for use with a medical device has an antenna embedded therein for performing radio communication with an external device situated outside the body. Such a capsule structure may undermine the quality of communication.

Accordingly, there is a need for a capsule that can maintain satisfactory communication conditions for use with a medical device.

### SUMMARY OF THE INVENTION

It is a general object of the present invention to provide a capsule for medical use that substantially eliminates one or more problems caused by the limitations and disadvantages of the related art.

According to one embodiment, a capsule for medical use which is to be passed through a tract of a human or animal body includes a capsule-shape case, a wire antenna placed in a wrapped or folded state on at least a portion of an outer surface of the case, and a fixing member configured to fix the wire antenna in the wrapped or folded state, wherein the fixing member is configured to release the antenna inside the tract.

A capsule for medical use which is to be passed through a tract of a human or animal body includes a capsule-shape case, an antenna part made of a metal foil disposed on a surface of the case, and a ground part made of a metal foil disposed on the surface of the case.

According to at least one embodiment, a capsule that can maintain satisfactory communication conditions for use with a medical device is provided.

### BRIEF DESCRIPTION OF THE DRAWINGS

Other objects and further features of the present invention will be apparent from the following detailed description when read in conjunction with the accompanying drawings, in which:

FIG. 1 is a perspective view of a capsule for use with a medical device according to a first embodiment;

FIG. 2 is a cross-sectional view of the medical device capsule of the first embodiment;

FIGS. 3A through 3C are drawings illustrating the way an antenna part of the medical device capsule of the first embodiment extends itself inside a body;

FIGS. 4A and 4B are drawings illustrating a medical device capsule according to a second embodiment;

FIGS. 5A and 5B are drawings illustrating a medical device capsule according to a third embodiment;

FIGS. 6A and 6B are drawings illustrating a medical device capsule according to a fourth embodiment;

FIGS. 7A and 7B are drawings illustrating a medical device capsule according to a fifth embodiment;

FIGS. 8A through 8C are drawings illustrating a medical device capsule according to a sixth embodiment; and

FIGS. 9A through 9C are drawings illustrating a medical device capsule according to a seventh embodiment.

### DESCRIPTION OF THE PREFERRED EMBODIMENTS

In the following, a description will be given of embodiments of a capsule for use with a medical device.

### First Embodiment

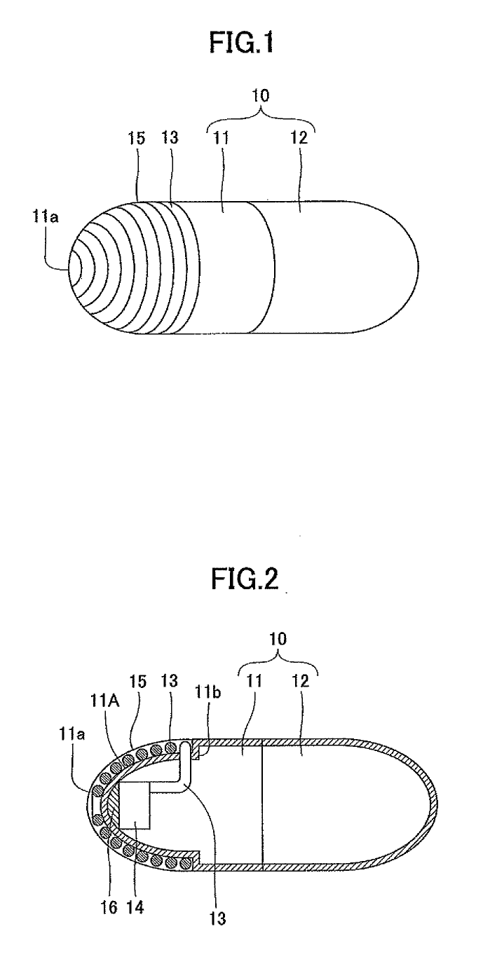

FIG. 1 is a perspective view of a capsule for use with a medical device according to a first embodiment.

A capsule 10 is a resin cylinder having ends thereof rounded into a hemispherical shape. The capsule 10 is 10 mm in length and 6 mm in diameter. The resin material constituting the capsule 10 may be an ABS (acrylonitrile butadiene styrene) resin, for example.

The capsule 10 is assembled by hermitically connecting a pair of capsule parts 11 and 12. An end 11a of the capsule part 11 has an antenna part 13 wrapped around its outer circumferential surface.

FIG. 2 is a cross-sectional view of the medical device capsule of the first embodiment.

The capsule part 11 has a retracted surface 11A on which the antenna part 13 is wrapped around.

The antenna part 13 has one end thereof inserted into a hole 11b provided in the capsule part 11 for connection with a transmitter 14 provided inside the capsule 10. The remainder of the antenna part 13 is wrapped such that the other end of the antenna part 13 reaches the ultimate point of the end 11a of the capsule part 11. The antenna part 13 may be made of copper.

The antenna part 13 is fixed by gelled gelatin 15 in such a shape that the antenna part 13 is wrapped around the outer circumferential surface of the capsule part 11. Namely, the antenna part 13 is fixed by the gelatin 15 serving as a fixing member while it is wrapped around the outer circumferential surface of the capsule part 11.

The length of the antenna part 13 matches the radio frequency that is employed for radio communication. For example, the length of the antenna part 13 is approximately 190 mm when the radio communication frequency is 400 MHz. Further, the length is approximately 94 mm for a radio frequency of 80 MHz, approximately 31 mm for a radio frequency of 2.4 GHz, and approximately 13 mm for a radio frequency of 5.8 GHz.

The thickness of the antenna part 13 may preferably be 0.05 mm, for example. If a UWB (Ultra Wide Band) frequency is used, the thickness may be increased to 0.1 mm to match such wide-band communication.

The antenna part 13 is configured to receive an electrical power from the transmitter 14 situated inside the capsule 10. Data is transmitted through the collaboration of the antenna part 13 with a ground part 16 disposed inside the end 11a of the capsule part 11.

The transmitter 14 is coupled to an imaging device (not illustrated) disposed inside the capsule 10, and is configured to transmit image data supplied from the imaging device to an external device situated outside the body.

It suffices for the gelatin 15 to be edible as a human swallows the capsule 10. The gelatin 15 is preferably configured to maintain the antenna part 13 in the wrapped state prior to being swallowed and to release the antenna part 13 from the wrapped state after being swallowed. In consideration of this, the composition and amount of the gelatin 15 are adjusted such that the gelatin 15 is dissolved while passing through the esophagus.

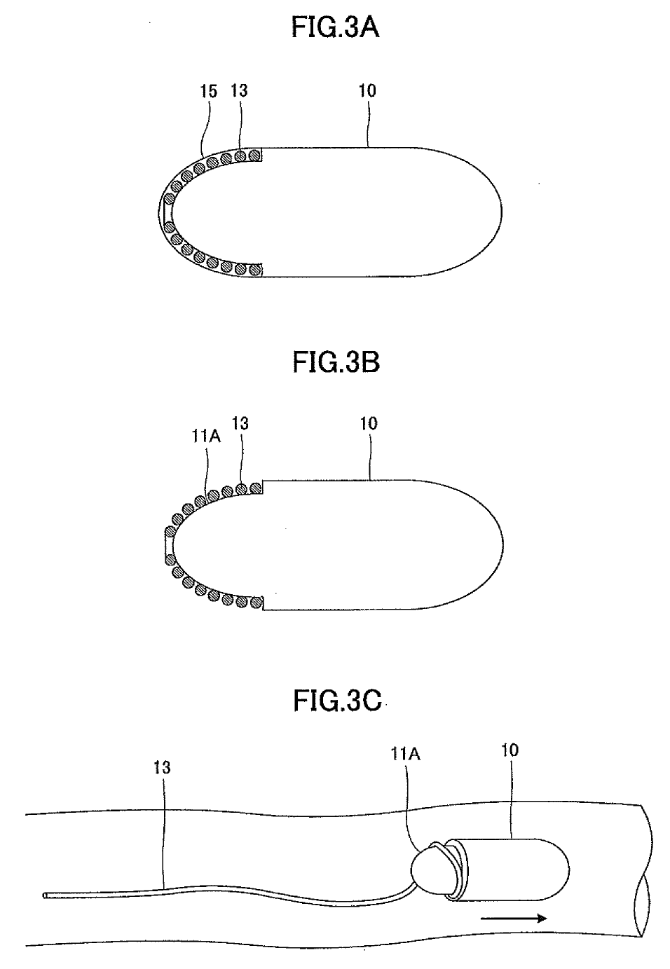

FIGS. 3A through 3C are drawings illustrating the way the antenna part 13 of the medical device capsule 10 of the first embodiment extends itself inside a body, A temporal sequence progresses in the following order: FIG. 3A, FIG. 3B, and FIG. 3C.

As FIGS. 3A through 3C are provided for the purpose of explaining the way the gelatin 15 is dissolved, the interior structure of the capsule 10 is omitted from the illustration.

FIG. 3A illustrates the state of the capsule 10 prior to being swallowed by a human or animal. In this state, the antenna part 13 is kept in the wrapped state by the gelled gelatin 15.

FIG. 3B illustrates the state of the capsule 10 immediately after being swallowed by a human or animal. In this state, gelatin 15 is almost all dissolved and gone, but the antenna part 13 is not yet released from the wrapped state.

FIG. 3C illustrates the state in which the gelatin 15 is all dissolved and gone, and the antenna part 13 is extended inside the intestine of the human or animal body. The capsule 10 is propelled to the right in the figure through the peristaltic movements of the intestine, so that the antenna part 13 is extended to trail behind the capsule 10 in its travel direction.

The occurrence of such extension of the antenna part 13 is not limited to inside the intestine, but may similarly occur inside the stomach or any other digestive tract.

When the capsule 10 is expelled to outside the body, the antenna part 13 is also expelled. The load on the human or animal body using the capsule 10 is about the same as the load imposed by a conventional medical device capsule.

As described above, the medical device capsule 10 of the first embodiment has the antenna part 13 extending inside the digestive tract of a human or animal body, so that its communication is not likely to be affected by an imaging device and the like disposed inside the capsule 10, thereby achieving satisfactory communication conditions.

The measurements of the capsule 10 and the antenna part 13 are not limited to the above-described values. The capsule 10 may have a length of 5 to 20 mm and a diameter of 5 to 10 mm.

Likewise, the antenna part 13 may have a length of 13 to 190 mm and a thickness of 0.05 to 0.1 mm.

The above description has been given with reference to an example in which the antenna part 13 is made of copper. This is not a limiting example, and the antenna part 13 may be made of an alloy including copper (Cu), nickel-chrome (Ni—Cr), and an amorphous alloy inclusive of ferrite. As an alloy inclusive of amorphous ferrite, an amorphous Fe—Si—B alloy (Fe: iron, Si: silicon, B: boron) may be used.

The amorphous Fe—Si—B alloy has a shape memory property. The antenna part 13 may be given a memorized shape such that the antenna part 13 returns to an extended state at 30 degrees Celsius close to the normal human body temperature.

Nickel-chrome has a high thermal conductivity. In consideration of this, a small amount of electric current may be applied to the antenna part 13 when the capsule 10 is expected to be at a proper position inside the body. This results in the amorphous Fe—Si—B alloy being reliably extended inside the body.

Although the above description has been provided with reference to an example in which the gelatin 15 is used as a fixing member, the fixing member is not limited to the gelatin 15. The fixing member may be of any mechanism as long as it can keep the antenna part 13 in the wrapped state prior to swallowing the capsule 10 and can release the antenna part 13 from the wrapped state by dissolving, breaking apart, or falling off inside the digestive tract after the capsule 30 is swallowed.

### Second Embodiment

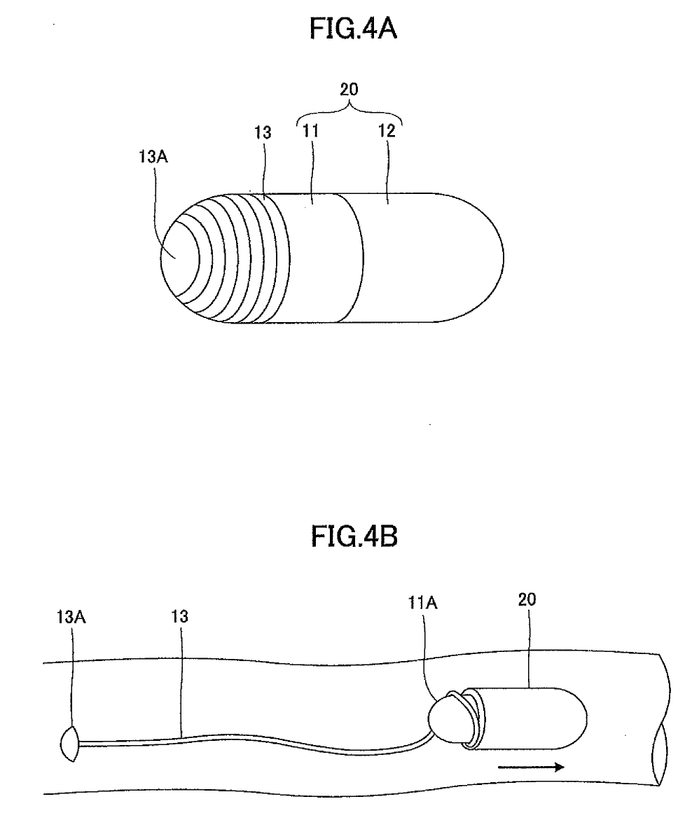

FIGS. 4A and 4B are drawings illustrating a medical device capsule according to a second embodiment. FIG. 4A is a perspective view, and FIG. 4B is a view illustrating a state in which the antenna is extended. A capsule 20 for use with a medical device according to the second embodiment differs from that of the first embodiment in that an anchor is provided at the tip of the antenna part 13. The remaining parts are the same as those of the first embodiment. The same elements are referred to by the same numerals, and a description thereof will be omitted.

The capsule 20 of the second embodiment has an anchor 13A at the tip of the antenna part 13. The anchor 13A serves to further extend the antenna part 13 to improve the communication conditions inside the human or animal body.

The anchor 13A may be a foldable weight made of resin, which is 3 mm long, 6 mm wide, and 6 mm high in a folded state. As the anchor 13A detaches from the capsule 20, the antenna part 13 extends.

As shown in FIG. 4A, the anchor 13A forms an additional projection at the end of the capsule 20 prior to being swallowed, compared to the capsule 10 of the first embodiment. The capsule 20 may still be 10 mm long and 6 mm wide, so that the easiness to swallow is about the same between the capsule 10 and the capsule 20.

As illustrated in FIG. 4B, the anchor 13A unfolds inside the intestine to function as resistance against the traveling movement of the capsule 20, thereby helping to extend the antenna part 13.

Accordingly, the medical device capsule 20 of the second embodiment has the anchor 13A assisting the antenna part 13 in extending inside the digestive tract of a human or animal body, so that its communication is not likely to be affected by an imaging device and the like disposed inside the capsule 10, thereby achieving satisfactory communication conditions.

The mounting location of the anchor 13A is not limited to the tip of the antenna part 13. The anchor 13A may be attached to any position along the extension of the antenna part 13.

The material of the anchor 13A is not limited to resin, and may be made of gelatin that is dissolvable inside the body. In such a case, the composition and amount of the gelatin are selected such that the anchor 13A remains to exist and continues to function until the very last minute, immediately prior to expulsion from the body, unlike those of the gelatin 15 that serves to fix the antenna part 13 prior to being swallowed.

### Third Embodiment

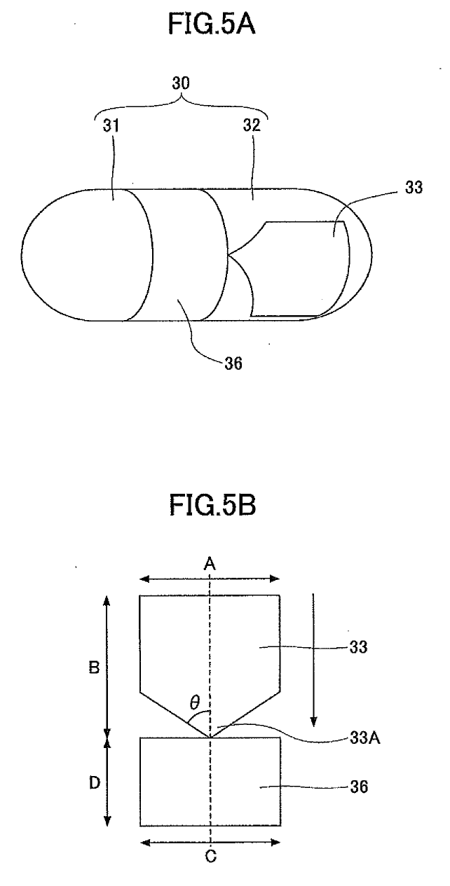

FIGS. 5A and 5B are drawings illustrating a medical device capsule according to a third embodiment. FIG. 5A is a perspective view of an antenna 33 attached to a capsule 30, and FIG. 5B is a view illustrating the antenna 33 before being attached to the capsule 30.

As shown in FIG. 5A, the capsule 30 is assembled by hermetically connecting a pair of capsule parts 31 and 32. The shape of the capsule part 32 is the same as the shape of the capsule part 12 of the first embodiment. Unlike the capsule part 11 of the first embodiment, however, the capsule part 31 does not have the retracted surface 11A formed thereon. The capsule 30 is 20 mm in length and 6 mm in diameter.

The antenna part 33 and ground part 36 are pasted onto the capsule parts 32 and 31, respectively, and are covered with resin coating. The resin that coats the antenna part 33 and the ground part 36 may be polyimide, for example.

As shown in FIG. 5B, the antenna part 33 is a copper foil having a home-plate shape in its plan view, and the ground part 36 is a copper foil having a rectangular shape in its plan view.

With respect to the antenna part 33, length A is 18 mm, and width B is 8 mm, with an angle θ relative to a centerline being 63 degrees. An electrical power is supplied to the antenna part 33 at a supply point 33A situated at the apex of the home-plate shape.

The ground part 36 may have a length C of 18 mm (equal to the length of the antenna part 33) and a width D of 8 mm.

The shape of the antenna part 33 and ground part 36 is designed such that UWE communication is attainable. This antenna has an excellent communication capacity that achieves a VSWR (voltage standing wave ratio) of 2.0 or less for a range of 3.1 GHz to 10.6 GHz.

Such an antenna part 33 and ground part 36 are pasted to the surfaces of the capsule parts 32 and 31, respectively, as illustrated in FIG. 5A. As in the case of the capsule of the first embodiment, a transmitter is provided to transmit image data at high speed to an external apparatus situated outside the body as an imaging device inside the capsule takes pictures.

The antenna part 33 is coupled to the embedded transmitter through a hole as in the case of the capsule 10 of the first embodiment, and this transmitter supplies electrical power to the supply point 33A.

In the third embodiment, the transmitter corresponding to the transmitter 14 of the first embodiment is configured to perform UWB (Ultra Wide Band) communication.

As described above, the medical device capsule 30 of the third embodiment can perform UWB communication between inside the digestive tract of a human or animal body and an external apparatus situated outside the body.

Because UWB communication is employed, the position of the capsule 30 inside the digestive tract can be identified by measuring the distances from a plurality of external apparatuses situated outside the body.

Further, not only the antenna part 33 but also the ground part 36 is disposed on the outer surface of the capsule 30. Such a configuration reduces the influence of noise generated by the imaging device or the like provided inside the capsule 30, thereby achieving satisfactory communication conditions.

The measurements of the capsule 30, the antenna part 33, and the ground part 36 are not limited to the above-described values. The capsule 30 may have a length of 5 to 20 mm and a diameter of 5 to 10 mm.

With respect to the antenna part 33, the length A may be 15 to 30 mm, and the width B may be 2 to 8 mm, with the angle θ relative to the center line being 45 to 70 degrees. For the ground part 36, the length C may be 15 to 30 mm, and the width D may be 2 to 8 mm.

### Fourth Embodiment

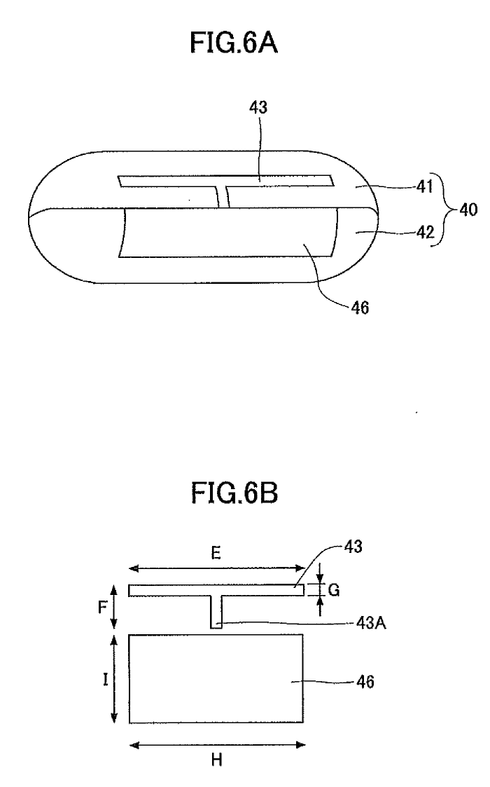

FIGS. 6A and 6B are drawings illustrating a medical device capsule according to a fourth embodiment. FIG. 6A is a perspective view of an antenna 43 attached to a capsule 40, and FIG. 6B is a view illustrating the antenna 43 before being attached to the capsule 40.

As shown in FIG. 6A, the capsule 40 is assembled by hermetically connecting a pair of capsule parts 41 and 42. The capsule parts 41 and 42 of the fourth embodiment are divided in a different direction than the capsule parts of the first through third embodiments. Namely, a dividing line extends in the longitudinal direction of the capsule 40. No retracted surface is provided. The capsule 40 is 10 mm in length and 6 mm in diameter.

The antenna part 43 and ground part 46 are pasted onto the capsule parts 41 and 42 of the capsule 40, respectively, and are covered with resin coating. This configuration is similar to that of the third embodiment. The resin that coats the antenna part 43 and the ground part 46 may be polyimide, for example.

As shown in FIG. 6B, the antenna 43 is a copper foil having a T-letter shape in its plan view, and the ground part 46 is a copper foil having a rectangular shape in its plan view.

With respect to the antenna part 43, length E is 8 mm, and width F is 3 mm, with width G being 1 mm. An electrical power is supplied to the antenna part 43 at a supply point 43A situated at the tip of the T-letter shape.

The ground part 46 may have a length H of 8 mm (equal to the length of the antenna part 43) and a width 1 of 10 mm.

The shape of the antenna part 43 and ground part 46 is designed such that UWB (Ultra Wide Band) communication is attainable. This antenna has an excellent communication capacity that achieves a VSWR (voltage standing wave ratio) of 2.0 or less for a range of 3 GHz to 10 GHz or 6 GHz to 20 GHz.

Such an antenna 43 and ground part 46 are pasted to the surfaces of the capsule parts 42 and 41, respectively, as illustrated in FIG. 6A. As in the case of the capsule of the first embodiment, a transmitter is provided to transmit image data at high speed to an external apparatus situated outside the body as an imaging device inside the capsule takes pictures.

The antenna 43 is coupled to the embedded transmitter through a hole as in the case of the capsule 10 of the first embodiment, and this transmitter supplies an electrical power to the supply point 43A.

In the fourth embodiment, the transmitter corresponding to the transmitter 14 of the first embodiment is configured to perform UWB (Ultra Wide Band) communication.

As described above, the medical device capsule 40 of the fourth embodiment can perform UWB communication between inside the digestive tract of a human or animal body and an external apparatus situated outside the body.

Further, not only the antenna 43 but also the ground part 46 is disposed on the outer surface of the capsule 40. Such a configuration reduces the influence of noise generated by the imaging device or the like provided inside the capsule 40, thereby achieving satisfactory communication conditions.

The measurements of the capsule 40, the antenna part 43, and the ground part 46 are not limited to the above-described values. The capsule 40 may have a length of 5 to 20 mm and a diameter of 5 to 10 mm.

With respect to the antenna part 43, the length E may be 4 to 8 mm, and the width F may be 1 to 4 mm, with the width G being 0.5 mm. For the ground part 46, the length H may be 4 to 8 mm, and the width I may be 4 to 10 mm.

### Fifth Embodiment

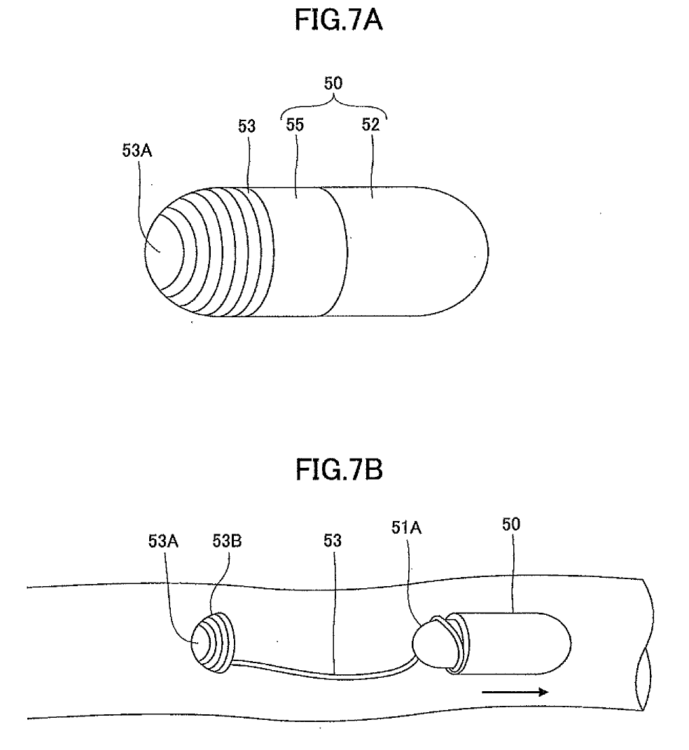

FIGS. 7A and 7B are drawings illustrating a medical device capsule according to a fifth embodiment. FIG. 7A is a perspective view of an antenna 53 attached to a capsule 50, and FIG. 7B is a view illustrating the antenna 53 in its extended state.

The medical device capsule 50 of the fifth embodiment includes capsule parts 55 and 52. An anchor 53A is provided at the tip of the antenna part 53, and a tip portion 53B of the antenna part 53 is adhered to the anchor 53A. In this manner, the capsule 50 for medical use differs from that of the second embodiment in that the tip portion 53B of the antenna part 53 is not extended but adhered to the anchor 53A provided at the tip of the antenna part 53. Namely, a portion of the wire of the antenna part 53 is kept adhered to the anchor 53A in its wrapped state.

As the tip portion 53B of the antenna part 53 is adhered to the anchor 53A, it is easier to extend the antenna part 53 as shown in FIG. 7B.

Further, the antenna tip portion 53B is kept in its wrapped state without being unfolded, thereby reducing a total length of the antenna extension. For example, the length of the antenna part 53 is approximately 190 mm when the radio communication frequency is 400 MHz. With the provision of the adhered tip portion 53 in the medical use capsule 50 of the fifth embodiment, the total length of the antenna extension may be reduced to 60 mm or 90 mm, which is approximately ⅓ or ½ of the original length.

### Sixth Embodiment

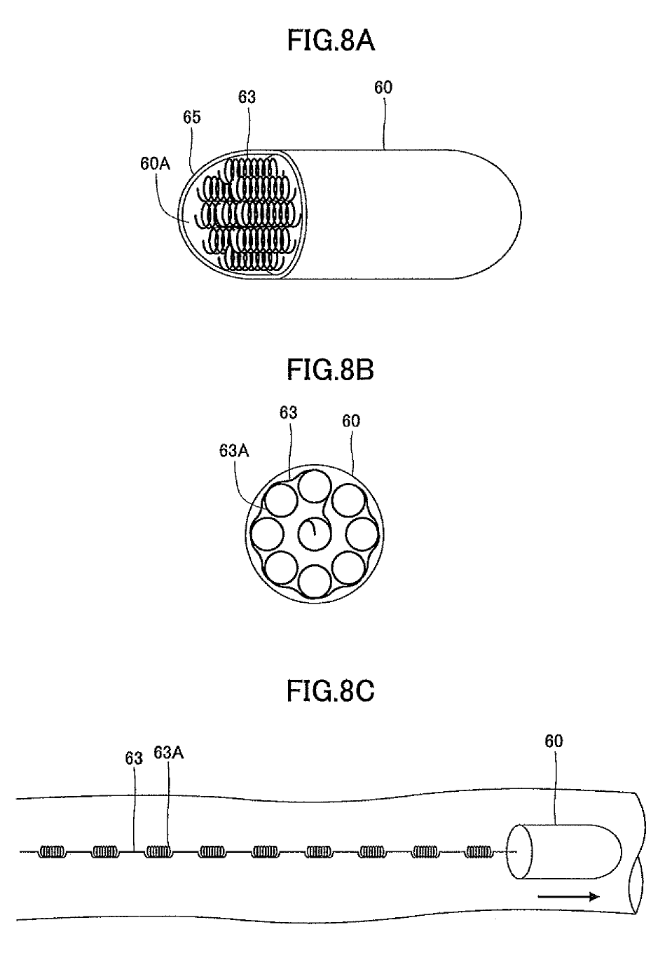

FIGS. 8A through 8C are drawings illustrating a medical device capsule according to a sixth embodiment. FIG. 8A is a partial cross-sectional view of a capsule as viewed from a lateral direction. FIG. 8B is a cross-sectional view of the capsule as viewed from its back. FIG. 8C is a view illustrating a state in which an antenna is extended. A capsule 60 for use with a medical device according to the sixth embodiment differs from that of the first embodiment in the structure of an antenna part 63.

As illustrated in FIGS. 8A and 8B, the antenna part 63 of the medical device capsule 60 of the sixth embodiment has nine helical parts 63A, each of which is a coil of the antenna wire. The antenna part 63 is a single conductive wire including the nine helical parts 63A.

The antenna part 63 is accommodated in a storage area 60A located at a tailing end of the capsule 60 such that the helical parts 63A and the remaining straight-line part are folded together. The antenna part 63 stored in the storage area 60A is fixed by gelled gelatin 65 as in the case of the medical device capsule 10 of the first embodiment.

When the medical device capsule 60 enters the body, the gelatin is dissolved, so that the antenna part 63 extends as shown in FIG. 8C. The helical parts 63A retain their coil shape.

In this manner, the total length of the antenna part 63 in its extended state is reduced due to the coils of the helical parts 63A, which suppress the extension of the antenna part 63. The capsule 60 for use with a medical device according to the sixth embodiment is advantageous especially when the length of the antenna part 63 is preferably shorter than otherwise.

Moreover, the helical parts 63A create electromagnetic induction. The magnetic field generated by this electromagnetic induction may be detected by an antenna or radar situated outside the body to detect the position of the capsule 60 inside the digestive tract.

### Seventh Embodiment

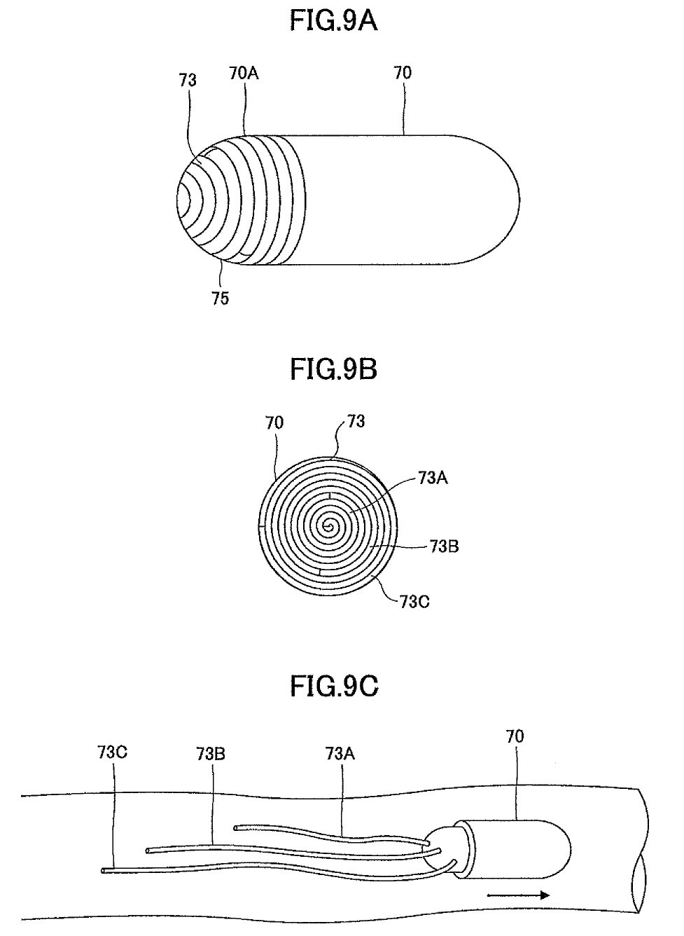

FIGS. 9A through 9C are drawings illustrating a medical device capsule according to a seventh embodiment. FIG. 9A is a perspective view, and FIG. 9B is a back view. FIG. 9C is a view illustrating a state in which the antenna is extended. A capsule 70 for use with a medical device according to the sixth embodiment differs from that of the first embodiment in the structure of an antenna part 73.

As illustrated in FIGS. 9A and 9B, the antenna part 73 of the medical device capsule 70 of the seventh embodiment has three antenna portions 73A, 73B, and 73C. The antenna portions 73A, 73B, and 73C each have different bandwidth characteristics.

The antenna portion 73A is a 2.4-GHz-band antenna, the antenna portion 73B is an 800-MHz-band antenna, and the antenna portion 73C is a 400-MHz-band antenna. The length of each of these antenna portions is designed to be approximately ¼ of the corresponding band wavelength. The antenna portion 73A is 31.2 mm long because the wavelength of a 2.4-GHz radio wave is 125 mm.

Similarly, the antenna portion 73B is 90 mm long because the wavelength of an 800-MHz radio wave is 370 mm. Further, the antenna portion 73C is 190 mm long because the wavelength of a 400-MHz radio wave is 750 mm.

The three antenna portions 73A, 73B, and 73C are accommodated in a storage area 70A situated at a trailing end of the medical device capsule 70 such that they are coiled in a spiral shape in its plan view. The antenna portions 73A, 73B, and 73C accommodated in the storage area 70A are fixed by gelled gelatin 75.

When the medical device capsule 70 enters the body, the gelatin is dissolved, so that the antenna portions 73A, 73B, and 73C extend as shown in FIG. 9C.

The medical device capsule 70 described above can utilize three different frequency bands, thereby being able to efficiently transmit data to outside the body. The capsule 70 for medical use according to the seventh embodiment is advantageous especially when the number of data types and/or the amount of data are large.

The descriptions of medical device capsules of exemplary embodiments have been provided heretofore. The present invention is not limited to these embodiments, but various variations and modifications may be made without departing from the scope of the present invention.

The present application is based on Japanese priority applications No. 2008-066549 filed on Mar. 14, 2008 and No. 2008-208534 filed on August 13, 2008, with the Japanese Patent Office, the entire contents of which are hereby incorporated by reference.

What is claimed is:

1. A capsule for medical use which is to be passed through a tract of a human or animal body, comprising:
a capsule-shape case;
a wire antenna placed in a wrapped or folded state on at least a portion of an outer surface of the case; and
a fixing member configured to fix the wire antenna in the wrapped or folded state,
wherein the fixing member is configured to release the antenna inside the tract.

2. The capsule as claimed in claim 1, wherein the antenna has an anchor attached to a tip thereof or at a halfway point along a longitudinal extension thereof.

3. The capsule as claimed in claim 2, wherein a portion of the wire antenna is wrapped around the anchor in a fixed manner.

4. The capsule as claimed in claim 1, wherein the fixing member includes gelatin that is dissolvable inside the tract.

5. The capsule as claimed in claim 1, wherein the antenna includes one or more helical parts, each of which is a coil made by winding a portion of the wire antenna.

6. The capsule as claimed in claim 1, wherein the antenna includes a plurality of antenna parts each having a different frequency band.

7. The capsule as claimed in claim 1, wherein the antenna is made of a shape memory alloy.

8. A capsule for medical use which is to be passed through a tract of a human or animal body, comprising:
a capsule-shape case;
an antenna part made of a metal foil disposed on a surface of the case; and
a ground part made of a metal foil disposed on the surface of the case.

9. The capsule as claimed in claim 8, wherein the case includes a pair of case parts that are coupled to each other to form a capsule shape, and wherein the antenna part is disposed on a surface of one of the case parts, and the ground part is disposed on a surface of another one of the case parts.

10. The capsule as claimed in claim 8, wherein the antenna part is a home-plate shape or T-letter shape in a plan view thereof.
